(19) United States
(12) Patent Application Publication
(10) Pub. No.: US 2008/0108079 A1
(43) Pub. Date: May 8, 2008

(54) GENES ASSOCIATED WITH COPD

(76) Inventor: Stephanie Chissoe, Durham, NC (US)

Correspondence Address: GLAXOSMITHKLINE;Corporate Intellectual Property, P. O. Box 13398, Five Moore Drive, Research Triangle Park, NC, 27709-3398, US

(21) Appl. No.: 11/933,476

(22) Filed: Nov. 1, 2007

Related U.S. Application Data
(60) Provisional application No. 60/864,683, filed on Nov. 7, 2006.

(51) Int. Cl. C12Q 1/68

(52) U.S. Cl. 435/6

(57) ABSTRACT

A method of screening a small molecule compound for use in treating COPD, comprising screening a test compound against a target selected from the group consisting of the gene products encoded by CELSR3, CHRNA5-THRU-CHRNB4, GPR55, LGR8, PMPCB, SENP1, UCHL1, UQCRC1, BRD2, CCK, HTR6, KCNK3, MBTPS2, NCOA6, PRSS7, SMO, THRA, or NR1D1, where activity against said target indicates the test compound has potential use in treating COPD.

### CROSS-REFERENCE TO RELATED APPLICATIONS

This application claims priority to U.S. provisional patent application No. 60/864,683 filed Nov. 7, 2006.

### FIELD OF THE INVENTION

The present invention relates to identification of genes that are associated with Chronic Obstructive Pulmonary Disease (COPD) and to screening methods to identify chemical compounds that act on those targets for the treatment of COPD or its associated pathologies.

### BACKGROUND OF THE INVENTION

The purpose of the present study was to identify genes coding for tractable targets that are associated with COPD, to develop screening methods to identify compounds that act upon such targets, and to develop such compounds as medicines to treat COPD and its associated pathologies.

Chronic Obstructive Pulmonary Disease (COPD) is a complex disorder characterized by chronic airflow obstruction, which is slowly progressive and mostly irreversible to current medical treatment, including bronchodilators (Pride et al, 1998).

COPD includes chronic bronchitis and emphysema provided there is airflow obstruction. Chronic bronchitis is defined as the presence of chronic productive cough for at least 3 months in each of 2 successive years in a patient in whom other causes of cough and sputum production have been excluded. Emphysema is defined anatomically as abnormal permanent enlargement of airspaces distal to the terminal bronchioles, accompanied by destructive changes of their walls and without obvious fibrosis.

The hallmark of COPD is the abnormal decline in lung function. Identifying causes and risk factors for the decline in lung function are the key to identifying the etiology of COPD. As the decline is slow and technical variability in lung function measurements is large, large population-based studies with a long follow-up time are needed.

According to the World Health Organization (WHO)-sponsored Global Burden of Disease Study COPD is expected to be a major public health problem of the future. In the United States, COPD is the fourth leading cause of death and a leading cause of morbidity, affecting 14 million persons in 1995 and producing 91,800 deaths (Feinleib et al, 1989). In the UK, chronic bronchitis and emphysema are estimated to affect 1 million people (overall prevalence of 1%, prevalence of 2% in men aged 45 to 65 years and 7% in men aged over 75 years). COPD is the third most common cause of death in the elderly, overall accounting for 5.4% of male deaths and 3.2% of female deaths (Office for National Statistics, 1993-1994).

The aetiology of COPD is likely to be influenced by multiple genetic and/or environmental factors. Cigarette smoking is the major known environmental risk factor for the development of COPD. Although the dose-response relationship between cigarette smoking and pulmonary function is well established, there is considerable variability in the degree of airway obstruction that occurs in response to smoking (Burrows et al, 1977).

The low percentage of variation in pulmonary function explained by smoking (approximately 15%) and the presence of persons with early-onset, severely reduced pulmonary function suggest that individuals may vary in their genetic susceptibility to the effects of smoking (Silverman et al, 1996). Additional causal risk factors of COPD include outdoor pollution and occupational exposures. Contributing risk factors include social status/poverty, respiratory infections, low birth weight, childhood respiratory illness, weight loss and nutrition (e.g. anti-oxidants) (Rijcken et al, 1996).

Currently, measures of lung function, most notably FEV1 (Force expiratory Volume in one second) and FEV1/FVC (Force expiratory Volume in one second/Forced Vital Capacity), are commonly used in the diagnosis and prognosis in COPD. In addition, it is the main endpoint in clinical trials. More recently, health status questionnaires and frequency and severity of exacerbations have been used to measure efficacy of new therapies. Repeat measurements over time are required to achieve significant indication of the progression of disease and the efficacy of therapeutic intervention (Berge et al, 2000).

### SUMMARY OF THE INVENTION

A first aspect of the present invention is a method for screening small molecule compounds for use in treating COPD, by screening a test compound against a target selected from the group consisting of gene products encoded by CELSR3, CHRNA5-THRU-CHRNB4, GPR55, LGR8, PMPCB, SENP1, UCHL1, UQCRC1, BRD2, CCK, HTR6, KCNK3, MBTPS2, NCOA6, PRSS7, SMO, THRA, and NR1D1. Activity against said target indicates the test compound has potential use in treating COPD.

### DETAILED DESCRIPTION

The present inventors tested genes that encode for potential tractable targets to identify genes that are associated with the occurrence of COPD and to provide methods for screening to identify compounds with potential therapeutic effects in COPD. An assessment of COPD data was carried out with a pooled data set of all 925 Caucasian cases and 937 Caucasian controls collected from Norway. The cases and controls were selected from a single centre at Haukeland Hospital, University of Bergen. Allelic and genotypic frequencies for the 6,836 Single Nucleotide Polymorphisms (SNPs) in 1,855 genes were contrasted between the cases and controls. In addition, gene-based permutation analyses were performed to account for the variable number of SNPs per gene. On the basis of these analyses, 8 genes or loci were identified as being significantly associated with COPD: CELSR3, CHRNA5-THRU-CHRNB4, GPR55, LGR8, PMPCB, SENP1, UCHL1, and UQCRC1. These genes all have a gene-based permutation P≦0.005 in the pooled data set. Likewise, an additional 10 genes showed statistical significance in the pooled data set with a permutation P>0.005 but <0.01. These genes are BRD2, CCK, HTR6, KCNK3, MBTPS2, NCOA6, PRSS7, SMO, THRA, and NR1D1.

As used, herein, a ‘tractable target’ or ‘druggable target’ is a biological molecule that is known to be responsive to manipulation by small molecule chemical compounds, e.g., can be activated or inhibited by small molecule chemical compounds. Classes of ‘tractable targets’ include, but are not limited to, 7-transmembrane receptors (7™ receptors), ion channels, nuclear receptors, kinases, proteases and integrins.

An aspect of the present invention is a method for screening small molecule compounds for use in treating COPD, by screening a test compound against a target selected from the group consisting of proteins encoded by the genes CELSR3, CHRNA5-THRU-CHRNB4, GPR55, LGR8, PMPCB, SENP1, UCHL1, UQCRC1, BRD2, CCK, HTR6, KCNK3, MBTPS2, NCOA6, PRSS7, SMO, THRA, and NR1D1. Activity against said target indicates the test compound has potential use in treating COPD. Activity may be enhancing (increasing) the biological activity of the gene product, or diminishing (decreasing) the biological activity of the gene product.

### EXAMPLE 1

### Subjects and Methods

Sample Set

The sample set consisted of 979 Caucasian cases and 980 Caucasian controls of which 925 Caucasian cases and 937 Caucasian controls were used in the study. All subjects were collected through the Department of Thoracic Medicine, Haukeland Hospital, Bergen, Norway and gave informed consent for the use of their DNA in this study. The cases and controls were recruited concurrently from December 2002-December 2004. Caucasian is defined as having 3 of 4 grandparents self-reported as Caucasian.

To be enrolled as a case the participant had to have a lung function severity of at least moderate IIA according to Global Initiative for Chronic Obstructive Lung Disease (GOLD) Criteria: FEV1/FVC<0.7, FEV1<80% (after bronchodilator medication).
[Reference: Global Strategy for the Diagnosis, Management, and Prevention of Chronic Obstructive Pulmonary Disease: NHLBI/WHO Workshop]
   In addition cases must have had no evidence of severe 1-antitrypsin deficiency (ZZ, Z Null, Null-Null, or SZ) assessed by Alpha 1 Antiytrypsin protease inhibitor (PI) type determined using isoelectric focusing.

To meet the criteria for a control the participant must not have been diagnosed with COPD: FEV1/FVC=0.7, FEV1=80% (after bronchodilator medication). Both cases and controls must have been at least 40 years of age at the time of enrollment and must have been a current or ex-cigarette smoker with a minimum of 2.5 pack years smoking exposure.

Target Genes

Relatively few human proteins, approximately a hundred in total, are considered to be suitable targets for effective small molecule drugs. It was considered reasonable to include all the members of these families for which a sequence was available. At the time, some of the genes were not exemplified in the public domain and were discovered through the analysis of expressed sequence tags or genomic sequence using a combination of sequence analysis. In addition, genes were selected because they were the targets of effective drugs even though they were not part of large protein families. Finally, disease expertise was employed to select genes whose involvement in COPD was either proven or suspected. Although over 2000 genes were selected in total, only 1,855 genes were analyzed due to attrition in SNP identification, primer design, genotyping and data quality control. Genes were named accordingly to NCBI ENTREZ Gene.

SNP Identification

The genes were automatically assembled and annotated with a region of the gene designated as 5′ and 3′, intron and exon. SNPs were mapped using BLAST to the manually curated genomic sequences. The SNPs were selected up to 10 kb from the start and stop sites of the transcripts with an average intermarker distance of 30 Kb. SNPs with a minor allele frequency (MAF)>5% were selected, but, all known coding SNPs were included irrespective of MAF. Approximately 10% of genes had fewer than 6 SNPs and these were subjected to SNP discovery using 24 primer pairs per gene to amplify 12 DNAs selected from Coriell Cell Repository of female CEPH cell-line samples. (CEPH refers to the Centre d'Etude du Polymorphisme Humain, which collected Northern European DNA samples.) For all of the discovered SNPs a minor allele frequency was determined using the FAST (Flow Accelerated SNP Typing) (Taylor et al, 2001) technology using multiplex PCR coupled with Single Base Chain Extension (SBCE) and Amplifluor genotyping. A marker selection algorithm was used to remove highly correlated SNPs to reduce the genotyping requirement while maintaining the genetic information content throughout the regions (Meng et al, 2003).

Sample Preparation and Genotyping DNA was isolated from whole blood using a basic salting-out procedure. Samples were arrayed and normalized in water to a standard concentration of 5 ng/ul. Twenty nanogram aliquots of the DNA samples were arrayed into 96-well PCR plates. For purposes of quality control, 3.4% of the samples were duplicated on the plates and two negative template control wells received water. The samples were dried and the plates were stored at −20° C. until use. Genotyping was performed by a modification of the single base chain extension (SBCE) assay previously described (Taylor et al. 2001). Assays were designed by a GlaxoSmithKline in-house primer design program and then grouped into multiplexes of 50 reactions for PCR and SBCE. Following genotyping, the data was scored using a modification of Spotfire Decision Site Version 7.0 Genotypes passed quality control if: a) duplicate comparisons were concordant, b) negative template controls did not generate genotypes and c) more than 80% of the samples had valid genotypes. Genotypes for assays passing quality control tests were exported to an analysis database.

Data Handling

The GSK database of record for analysis-ready data is called SubjectLand. This database contains all genotypes, phenotypes (i.e. clinical data), and pedigree information, where applicable, on all subjects used in the analysis of data for these studies. SubjectLand does not maintain information regarding DNA samples, but is closely integrated with the sample tracking system to maintain the connection between subjects and their samples and phenotypic data at all times. All subjects gave informed consent for the use of their DNA and phenotypic data in this study. The analytical tools used in the analysis process described below interface directly with subject data in SubjectLand. This interface also archives the files used in analysis as well as the results.

Analysis

Only subjects with a subject type (SBTY) of case or control were analyzed. Subjects with a SBTY of affected family member or other SBTY values were excluded from analysis. Subjects were also excluded if he/she, either parent, or more than one grandparent were non-Caucasian as indicated by self-report. In addition, subjects were excluded if their putative gender was inconsistent with SNP genotypes on the X chromosome. Finally, subjects that genotyped on fewer than 75% of the SNPs in a given genotyping experiment were excluded from analysis.

Each marker was examined for Hardy-Weinberg equilibrium and minor allele frequency. Genotypic and allelic associations test were then performed, followed by identification of the risk allele and risk genotype using chi-square tests. An odds ratio and confidence interval of greater than 95% was calculated for the risk allele and risk genotype. Next, population stratification was evaluated by determining if the number of allelic and genotypic tests observed to be significant at a given threshold was inflated with respect to what would be expected under the null hypothesis of no association. In addition, linkage disequilibrium (LD) was examined to measure the association between alleles at different loci (Weir, 1996, pp. 109-110). Lastly, a permutation assessment was conducted to account for the variable number of SNPs per gene and yield a single permutation p-value per gene for the pooled analysis data set. Statistically significant genes were identified as those passing gene-based permutation thresholds. The empirical permutation p-value from the pooled data set was required to fall at or below 0.005 to be considered significantly associated with COPD. Further, since the weight of statistical evidence occurs on a continuum, genes with a p-value greater than 0.005 or less than or equal to 0.01 were also considered statistically significant.

Hardy Weinberg Equilibrium

Hardy Weinberg equilibrium (HWE) is a measure of the association between two alleles at an individual locus. A bi-allelic marker is in HWE if the genotype frequencies are p2, 2pq and q2 for the genotypes 1, 1; 1, 2; and 2, 2 where p and q are the frequencies of the 1 and 2 alleles, respectively. The departure from HWE was tested using a Chi square test, by testing the difference between the expected (calculated from the allele frequencies) and observed genotype frequencies. A HWE permutation test was performed when the HWE chi-square p-value <0.05 and when at least one genotype cell had an expected count less than 5 (Zaykin et al, 1995). When these conditions exist, the HWE chi-square test may not be valid and a permutation test to assess departure from HWE is warranted. Markers failing HWE at p≦0.001 in controls were removed from the pooled analysis marker cluster used in association analyses. HWE failure may indicate a non-robust assay.

Minor Allele Frequency

For minor allele frequency, markers which were monomorphic were removed from the analysis marker cluster used in association analyses.

Allelic and Genotypic Test of Association

Testing for association in the study data was carried out using the ‘PROC FREQ’ fast Fisher's exact test (FET) procedure in the statistical software package SASv8.2. An exact test is warranted in situations when asymptotic assumptions are not met such as when the sample size is not large or when the distribution is sparse or skewed. Such situations occur for SNPs with rare minor allele frequencies where the number of expected cases and/or controls for the rare homozygote are less than 5. Under these conditions, the asymptotic results many not be valid and the asymptotic p-value may differ substantially from the exact p-value. The classic Fisher's Exact Test computes exact p-values by enumerating all tables as extreme as, or more extreme than, that observed. This direct enumeration approach is very time-consuming and only feasible for small problems. The fast Fisher's Exact test computes exact p-values for general R×C tables using the network algorithm developed by Mehta and Patel (1983). The network algorithm provides substantial advantage over direct enumeration and is rapid and accurate.

Tables I and II show the structure of the genotype and allele contingency tables, respectively.

TABLE I
Generic disease status by genotype contingency table.
Disease Status

| | | Case | Control | Total |
|---|---|---|---|---|
| Genotype | AA | n11 | n12 | n1. |
| | Aa | n21 | n22 | n2. |
| | aa | n31 | n32 | n3. |

| | | | |
|---|---|---|---|
| Total | n.1 | n.2 | N |

TABLE II
Generic disease status by allele contingency table.
Disease Status

| | | Case | Control | Total |
|---|---|---|---|---|
| Allele | A | 2n11 + n21 | 2n12 + n22 | 2n1. + n2. |
| | a | 2n31 + n21 | 2n32 + n22 | 2n3. + n2. |

| | | | |
|---|---|---|---|
| Total | 2n.1 | 2n.2 | 2N |

Risk Allele and Risk Genotype

The “risk allele” refers to the allele that appeared more frequently in cases than controls. The “risk genotype” was determined after identifying the genotype that had the largest chi-square value when compared against the other 2 genotypes combined in the genotypic association test. For example, if a SNP had genotypes AA, AG and GG, 3 chi-square tests were performed contrasting cases and controls: 1) AA vs AG+GG, 2) AG vs AA+GG and 3) GG vs AA+AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

Odds Ratios and Confidence Intervals

An odds ratio was constructed for the risk allele and risk genotype.
Odds ratio (OR)=(n11*n22)/(n12*n21)

where
n11=cases with risk genotype
n21=cases without risk genotype
n12=controls with risk genotype
n22=controls without risk genotype
   In order to avoid division or multiplication by zero, 0.5 was added to each cell in the contingency table (as recommended in “Statistical Methods for Rates and Proportions” by Fleiss, Ch 5.3 p. 64)

A 95% confidence interval for the odds ratio was also calculated as follows:

where
z=97.5th percentile of the standard normal distribution
v=[1/(n11)]+[1/(n12)]+[1/(n21)]+[1/(n22)].
   Evaluation of Population Stratification

In this assessment, cases and control frequencies were compared across a subset of relatively independent markers (markers in low LD) selected from the set of all markers analyzed. Since the vast majority of genes on the gene list are not associated with a specific disease, this constitutes a null data set. If the cases and controls are from the same underlying population, the expectation is to see 5% of the tests significant at the 5% level, 1% significant at the 1% level, etc. If, on the other hand, the cases and controls are from different populations, (for example, cases from Finland and controls from Japan), there would be an inflation in the proportion of tests significant across thresholds due to genetic differences between the two populations that are unrelated to disease. Inflation in the number of observed significant tests over a range of cut-points suggests that the case and control groups are not well matched. Consequently, the inflated number of positive tests may be due to population stratification rather than to association between the associated SNPs and disease.

The probability of ≧m observed number of significant tests out of n total tests at a cut-point p was calculated using the binomial probability as implemented in either S-PLUS or SAS.

With SAS PROBNML (p,n,m) computes the probability that an observation from a binomial (n,p) distribution will be less than or equal to m.

Linkage Disequilibrium

The LD between two markers is given by DAB=pAB-pApB, where pA is the allele frequency of A allele of the first marker, pB is the allele frequency of B allele of the second marker, and pAB is the joint frequency of alleles A and B on the same haplotype. LD tends to decline with distance between markers and generally exists for markers that are less than 100 kb apart

The SAS procedure PROC CORR was used to calculate r using the Pearson product-moment correlation. To determine whether significant LD existed between a pair of markers we made use of the fact that nr2 has an approximate chi square distribution with 1 df for biallelic markers. The significance level of pairwise LD was computed in SAS.

Permutation Assessment

The analysis of the observed un-permuted data led to a set of observed p-values for each gene. We defined min [obs(p)] as the minimum p-value derived from all tests of all SNPs within the gene for a given data set. The objective of this permutation test was to determine the significance of this minimum p-value in context of the number of SNPs analyzed number of tests conducted and the correlation between SNPs within each gene. The permutation process accounted for the multiple SNPs and tests conducted within a particular gene but it did not account for the total number of genes being analyzed.

Due to computational limitations, only those genes with a min [obs (p)] less than a threshold of 0.05 were assessed for significance using a permutation process. A maximum number of permutations, N, was conducted per gene (N=50,000 for pooled set; see below). However, this maximum number did not need to be conducted for every gene. For many genes far fewer permutations were sufficient to show that a gene was not significant at the threshold of interest and the permutation process for that gene was terminated early.

The following process was followed. For each permutation, affection status was shuffled among the cases and controls, maintaining the overall number of cases and number of controls in the observed data. The genetic data for each subject were not altered. For each permutation, all the SNPs within a gene were analyzed using allelic and genotypic association tests (same methods as employed with true, observed data). The p-value for the most significant test, min [sim (p)] was captured for each permutation. The permutations were repeated up to N times such that up to N min [sim (p)]'s were captured. Once the permutations were completed, the min [obs (p)] for each gene was compared against the distribution of min [sim (p)]. The proportion of min [sim (p)] that was less than the min [obs (p)] gave the empirical permutation p-value for that gene. This p-value was labelled perm (p).

The maximum number of iterations needed to accurately assess the permutation p-value depended on the threshold set for declaring significance. For example, in assessing permutation p-values below 0.05, 5000 permutations gave a 95% confidence interval (CI) of 0.044 to 0.056. This was not considered to be a tight enough estimate of the true permutation p-value. By assessing 50,000 permutations the 95% CI was narrowed considerably, to 0.48 to 0.52. The CIs for a range of permutation p-values and numbers of permutations are presented below.

| permP | 5000 CI | 10000 CI | 50000 CI |
|---|---|---|---|
| 0.05 | (0.044, 0.056) | (0.0457, 0.0543) | (0.048, 0.052) |
| 0.01 | (0.0072, 0.0128) | (0.008, 0.012) | (0.0091, 0.011) |
| 0.005 | (0.003, 0.008) | (0.0036, 0.0064) | (0.0044, 0.0056) |

Based on the above CI estimates, genes in the pooled data set with an obs (p)≦0.05 were assessed with a maximum of 50,000 permutations.

### EXAMPLE 2

### Results

Seventy-eight collected subjects were excluded from the study based on sample set quality control (QC) measures. Fifty-five were excluded for subject type, for ethnicity, 1 for gender inconsistency, 1 for other reasons, and 11 that genotyped on fewer than 75% of the SNPs. Controls are on average 10 years younger than the cases and have a smaller proportion of males (50% in controls compared to 61% in cases). Average smoking history (in pack years) was 12.8 years higher in cases than controls. Because controls are substantially younger and have far less smoking exposure than cases, a proportion of controls may turn out to be cases in waiting. Key demographic characteristics of the pooled data set are detailed in Table 1.

During SNP marker quality control, 64 SNPs were excluded due to Hardy-Weinberg Equilibrium (HWE). 33 SNPs were excluded because SNPs were monomorphic in cases and controls. 119 SNPs were excluded due to mapping issues. As a result, 6,836 SNPs were analyzed for association with COPD of which 6,749 had a gene assignment and 87 did not. In total 1,855 genes were analyzed: 1,787 autosomal, 68 X-linked. The mean number of SNPs per genes was 3.7 with a range of 1-53 SNPs per gene. See Table 2 for a summary SNP coverage of genes.

Detailed summaries of genotype counts across all genes and subjects analysed are given in Table 3 and Table 4. The apparent bimodal distribution seen in the tables reflect the staged genotyping process and the evolution of the gene list over time.

After gene-based permutation analysis, 8 genes were identified as having the strongest statistical evidence for genetic associated with COPD (Table 5). The set of genes reached a gene-based permutation P-value of <=0.005 in the pooled data set of all 925 cases and 937 controls. The 10 genes in Table 6 are the next best in terms of statistical evidence. These genes have a gene-based permutation P-value between 0.005 and 0.01.

The number of tests significant across various thresholds was not inflated beyond what is expected by chance (Table 7).

CELSR3, CHRNA5-THRU-CHRNB4, GPR55, LGR8, PMPCB, SENP1, UCHL1, UQCRC1, BRD2, CCK, HTR6, KCNK3, MBTPS2, NCOA6, PRSS7, SMO, THRA, and NR1D1 passed statistically significant gene-based permutation thresholds in the pooled data set. These genes have the strongest statistical evidence for association with COPD. Further, there was no evidence of population stratification based on the distribution of results.

However, it is possible that some of the associations are false positives. Statistical association between a polymorphic marker and disease may occur for several reasons. The marker may be a mutation that influences disease susceptibility directly or may be correlated with a mutation that influences disease susceptibility because the marker and disease susceptibility mutation are physically close to one another. Spurious association may result from issues such as confounding or bias although the study design attempts to remove or minimize these factors. The association between a marker and disease may also be due to chance.

The gene-wise type 1 error is the gene-based permutation p-value threshold used to identify the genes of interest. It also provides the false positive rate associated with each gene. Out of 1855 genes examine, an average of 9.3±3.0 would be expected to have a permutation p≦0.005 while 18.6±4.3 would be expected to have a permutation p≦0.01.

TABLE 1
Collections analysed

| | Cases | Controls |
|---|---|---|
| Case/control status | 925 | 937 |
| Male:Female (% male) | 565:360 (61.1%) | 468:469 (50.0%) |
| Mean AGE +/− sd | 65.6 +/− 10.1 | 55.5 +/− 9.7 |
| Mean Smoking history in Pack Years +/− sd | 32.4 +/− 20.1 | 19.6 +/− 13.9 |
| Mean Body mass index +/− sd | 25.4 +/− 4.9 | 26.5 +/− 4.0 |
| Mean Waist Hip Ratio +/− sd | 0.9 +/− 0.1 | 0.9 +/− 0.1 |
| Mean Fat free mass +/− sd | 67.8 +/− 18.6 | 66.5 +/− [symbol] |
| Mean pre brocho dialator (percent predicted) +/− sd | 46.9 +/− 17.1 | 90.9 +/− 9.4 |
| Mean post brocho dialator (percent predicted) +/− sd | 49.9 +/− 17.5 | 93.9 +/− 9.0 |
| Mean MAXFVC pre brocho dialator +/− sd | 2.9 +/− 1.0 | 4.1 +/− 0.9 |
| Mean MAXFVC post brocho dialator +/− sd | 3.0 +/− 1.0 | 4.1 +/− 0.9 |
| Mean Estimated alveolar volume +/− sd | 5.15 +/− 1.2 | 5.65 +/− 1.2 |
| Mean FEV1 pre brocho dialator (absolute value) +/− sd | 1.5 +/− 0.7 | 3.1 +/− 0.7 |
| Mean FEV1 post brocho dialator (absolute value) +/− | 1.6 +/− 0.7 | 3.2 +/− 0.7 |
| Mean Maximal ratio (FEV1/FVC) +/− sd | 0.5 +/− 0.1 | 0.8 +/− 0.0 |

TABLE 2
SNP coverage of genes in analysis marker cluster

| | 1 | 2 | 3 | 4-5 | 6-9 | 10+ | |
|---|---|---|---|---|---|---|---|
| | SNP | SNPs | SNPS | SNPs | SNPs | SNPs | Total |
| No. | 433 | 459 | 363 | 301 | 190 | 109 | 1,855 |
| genes | | | | | | | |

TABLE 3
Summary of genotype counts across SNPs

| Numbers of subjects genotyped | Number of markers |
|---|---|
| 1801-1862 | 4,077 |
| 1601-1800 | 888 |
| 1501-1600 | 28 |
| 1001-1500 | 0 |
| <1001 | 1,843 |

* These represent SNPs added to the gene list after the primary screen was completed

TABLE 4
Summary of genotype counts across subjects

| Numbers of SNPS genotyped | Number of subjects |
|---|---|
| 6001-6836 | 860 |
| 5501-6000 | 71 |
| 5001-5500 | 910 |
| 4501-5000 | 21 |

TABLE 5
Genes with Permutation P value greater than or equal to 0.005

| | | | | |
|---|---|---|---|---|
| | Permutation | | | |
| Region² | P-value | Gene | Target Class | Gene Description |

Permutation P <= 0.005.

| | | | | |
|---|---|---|---|---|
| CELSR3³ | 0.0042 | CELSR3 | 7TM | cadherin, EGF LAG seven-pass |
| | | | | G-type receptor 3 (flamingo |
| | | | | homolog, Drosophila) |
| | | SLC26A6 | TRANSPORTER | solute carrier family 26, member 6 |
| CHRNA5- | | CHRNA3 | ION_CHANNEL | cholinergic receptor, nicotinic, |
| THRU- | | | | alpha polypeptide 3 |
| CHRNB4³ | 8.00E−05 | CHRNA5 | ION_CHANNEL | cholinergic receptor, nicotinic, |
| | | | | alpha polypeptide 5 |
| | | CHRNB4 | ION_CHANNEL | cholinergic receptor, nicotinic, |
| | | | | beta polypeptide 4 |
| GPR55 | 0.0025 | GPR55 | 7TM | G protein-coupled receptor 55 |
| LGR8 | 0.0005 | LGR8 | 7TM | similar to G protein coupled |
| | | | | receptor affecting testicular |
| | | | | descent (H. sapiens) |
| PMPCB³ | 0.0006 | PMPCB | PROTEASE | peptidase (mitochondrial |
| | | | | processing) beta |
| | | ZRF1 | Unclassified | zuotin related factor 1 |
| SENP1 | 0.0022 | SENP1 | PROTEASE | sentrin/SUMO-specific protease |
| UCHL1 | 0.0018 | UCHL1 | PROTEASE | ubiquitin carboxyl-terminal |
| | | | | esterase L1 (ubiquitin |
| | | | | thiolesterase) |

¹Genes represent the set of genes that have reached a gene-based permutation P-value of <= 0.005 in the pooled data set of all 925 cases and 937 controls.
²Region is a label used to assign a 1:1 relationship between a SNP and a unique part of the genome. In most instances the region and gene are one in the same. However, in gene rich parts of the genome (where SNPs map to multiple genes), a region may include several genes.
³Some regions, in gene rich parts of the genome, have SNPs which map to several genes or have overlapping genes. The disease association may to be any one of these genes.

TABLE 6
Genes with Permutation P-value between 0.005 and 0.01

| | | | | |
|---|---|---|---|---|
| | Permutation | | | |
| Region² | P-value | Gene | Target Class | Gene Description |

0.005 < Permutation P <= 0.01

| | | | | |
|---|---|---|---|---|
| BRD2 | 0.0091 | BRD2 | KINASE | bromodomain containing 2 |
| CCK | 0.0054 | CCK | 7TM_LIGAND | cholecystokinin |
| HTR6 | 0.0073 | HTR6 | 7TM | 5-hydroxytryptamine (serotonin) receptor 6 |
| KCNK3 | 0.0093 | KCNK3 | ION_CHANNEL | potassium channel, subfamily K, member 3 |
| MBTPS2 | 0.0086 | MBTPS2 | PROTEASE | membrane-bound transcription factor |
| | | | | protease, site 2 |
| NCOA6³ | 0.0051 | NCOA6 | NR_COFACTOR | nuclear receptor coactivator 6 |
| | | TP53INP2 | Unclassified | chromosome 20 open reading frame 110 |
| PRSS7 | 0.0081 | PRSS7 | PROTEASE | protease, serine, 7 (enterokinase) |
| SMO | 0.0077 | SMO | 7TM | smoothened homolog (Drosophila) |
| THRA_NR1D1³ | 0.0086 | THRA | NR | thyroid hormone receptor, alpha |
| | | NR1D1 | NR | (erythroblastic leukemia viral (v-erb-a) |
| | | | | oncogene homolog, avian) |
| | | | | nuclear receptor subfamily 1, group D, |
| | | | | member 1 |

¹These genes have a gene-based permutation p between 0.005 and 0.01 in 925 cases and 937 controls.
²Region is a label used to assign a 1:1 relationship between a SNP and a unique part of the genome. In most instances the region and gene are one in the same. However, in gene rich parts of the genome (where SNPs map to multiple genes), a region may include several genes.
³Some regions, in gene rich parts of the genome, have SNPs which map to several genes or have overlapping genes. The disease association may to be any one of these genes.

TABLE 7
Assessment of Population Stratification

| | Total No. | | | | |
|---|---|---|---|---|---|
| Analysis | genotypic | Genotypic Association | | Allelic Association | |
| p-values = | or allelic | No. tests < | Binomial | No. tests < | Binomial |
| p | tests | p(m) | prob ≧ m | p(m) | prob ≧ m |
| P < 0.05 | 2,291 | 98 | 0.94062 | 107 | 0.74793 |
| P < 0.01 | 2,291 | 19 | 0.75783 | 29 | 0.08726 |
| P < 0.005 | 2,291 | 11 | 0.47502 | 14 | 0.18054 |
| P < 0.001 | 2,291 | 5 | 0.02942 | 2 | 0.40161 |
| P < 0.0005 | 2,291 | 2 | 0.10887 | 1 | 0.31761 |

### REFERENCES

Berge P S., Calverley P M., Jones P W., Spencer S., Anderson J A. Maslen T K. (2000) Randomised, double blind, placebo controlled study of fluticasone propionate in patients with moderate to severe chronic obstructive pulmonary disease: the ISOLDE trial. British Medical Journal 320(7245):1297-303.
Burrows B., Knudson R J., Cline M G., Lebowitz M D. (1977) Quantitative relationships between cigarette smoking and ventilatory function. American Review of Respiratory Diseases 115:195-205,
Feinleib M., Rosenberg H M., Collins J G., Delozier J E., Pokras R., Chevarley F M. (1989) Trends in COPD morbidity and mortality in the United States. American Review of Respiratory Diseases 140:S9-18.
Reference: Global Strategy for the Diagnosis, Management, and Prevention of Chronic Obstructive Pulmonary Disease: NHLBI/WHO Workshop:
Fleiss J, Levin B., Paik M C. (2003) Statistical Methods for Rates and Proportions. 3^{rd} Edition. Wiley.
Mehta, C. and Patel, N. (1983) A Network Algorithm for Performing Fisher's Exact Test in rXc contingency tables. Journal of the American Statistical Association 78:427-434.
Meng, Z. et al. (2003) Selection of Genetic Markers for Association Analyses, Using Linkage Disequilbrium and Haplotypes. American Journal of Human Genetics 71(1): 115-130.
Office for National Statistics. Mortality Statistics: cause, England and Wales. Series DH2 No. 21 London: Government Statistical Service, 1993 and 1994 (revised)
Pride N B, Vermeire P. (1998) Management of chronic obstructive pulmonary disease, Chapter 2: Definition and differential diagnosis. European Respiratory Monograph 3:2-5.
Rijcken B, Britton J. (1998) Epidemiology of chronic obstructive pulmonary disease. In: Postma D S, Siafakas N M (Eds.) Management of chronic obstructive pulmonary disease. European Respiratory Society Monograph 7:41-73.
Roses A D., Burns D K., Chissoe S., Middleton L., St Jean P., (2005) Disease-specific target selection: A Critical First Step Down the Right Road. Drug Discovery Today 10: 177-189.
Silverman E K., Speizer F E. (1996) Risk factors for the development of chronic obstructive pulmonary disease. Med Clinics North Amer 80:501-22.
Taylor J D., Briley D., Nguyen Q., Long K., Tannone M A., Li M S., Ye F., Afshari A., Lai E., Wagner M., Chen J., Weiner M P. (2001) Flow cytometric platform for high-single nucleotide polymorphism analysis. [Journal Article] Biotechniques. 30(3): 661-6, 668-9, Mar.
Weir, BS. (1996) Genetic Data Analysis II, Sinauer Associates, Inc., Sunderland, Mass., pp. 109-110.
Zaykin D V, Zhivotovsky L A, Weir B S (1995) Exact tests for association between alleles at arbitrary numbers of loci. Genetica 96:169-178.

1. A method of screening a small molecule compound for use in treating COPD, comprising screening a test compound against a target selected from the group consisting of the gene products encoded by CELSR3, CHRNA5-THRU-CHRNB4, GPR55, LGR8, PMPCB, SENP1, UCHL1, UQCRC1, BRD2, CCK, HTR6, KCNK3, MBTPS2, NCOA6, PRSS7, SMO, THRA, or NR1D1, where activity against said target indicates the test compound has potential use in treating COPD.
